(19) United States
(12) Patent Application Publication
(10) Pub. No.: US 2015/0065937 A1
(43) Pub. Date: Mar. 5, 2015

(54) Bandage

(71) Applicant: Me4Kidz LLC, Phoenix, AZ (US)

(72) Inventor: Pete Nassos, Phoenix, AZ (US)

(73) Assignee: Me4Kidz LLC, Phoenix, AZ (US)

(21) Appl. No.: 13/987,741

(22) Filed: Aug. 27, 2013

(51) Int. Cl. A61F 13/00

(52) U.S. Cl. CPC A61F 13/00059; A61F 2013/00153
USPC 602/52; 602/54

(57) ABSTRACT

A self-adhering bandage comprising: a carrier strip; adhesive layer disposed thereon; a gauze strip located approximately centrally on the carrier strip; a release paper covering the gauze strip and held in place by its adhering contact with the adhesive, thus forming a laminate; and an indicia incorporated in the adhesive layer that is transferable from the adhesive layer and a user's skin, when the bandage is removed from the user's skin.

### FIELD OF THE INVENTION

The present invention relates to a bandage. More specifically, the present invention relates to a bandage that, after removal, leaves indicia on the epidermis.

### BACKGROUND OF INVENTION

In protecting minor cuts, scratches, abrasions, and similar injuries, particularly on a temporary, self-treatment basis, the use of the so-called self-adhesive bandage is widespread. Bandages of the self-adhesive type basically involve a length of adhesive tape with a pad of gauze, or similar material, located in the center portion thereof, the whole being covered by a separable layer of “release” paper which preserves the sterility of the gauze, and renders the adhesive properties of the tape inoperative until the paper is removed prior to application of the bandage. Such bandages come in a variety of shapes and sizes and are both inexpensive and easy to apply, which helps to account for their widespread popularity. In the past these bandages have been monochrome and aesthetically boring.

More recently, manufacturers of bandages have taken to adding graphics onto the outer surface of the bandage in order to make them more appealing to the user. This is most common in bandages designed and marketed for use on small children.

### SUMMARY OF THE INVENTION

In light of the foregoing, therefore, it is a first aspect of this invention to provide a bandage of the self-adhesive type that incorporates a temporarily transferable image.

It is a further aspect of this invention to provide a bandage of the self-adhesive type that incorporates a carrier strip; an adhesive layer on the carrier strip; a gauze strip, centered on the carrier strip; and a removable release paper, wherein the adhesive layer incorporate an image that is transferable from the adhesive layer to a skin surface.

It is a still further aspect of this invention to provide a bandage of the self-adhesive type that incorporates a carrier strip; an adhesive layer on the carrier strip; a gauze strip, centered on the carrier strip; a polymer release/image layer on the adhesive layer and a removable release paper, wherein the polymer release/image layer incorporate an image that transfers from the adhesive layer to a skin surface when the bandage is removed.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE DRAWINGS

The invention will be more fully understood by referring to the following Detailed Description of Specific Embodiments in conjunction with the Drawings, of which:

FIG. 1 is a sectional view of a self-adhering bandage of the invention along line 1-1 of FIG. 2.

FIG. 2 is a top plan view of a self-adhering bandage of the invention.

FIG. 3 is an exploded view of one embodiment of the present invention.

FIG. 4 is a sectional view of a second embodiment of the self-adhering bandage of the invention along line 4-4 of FIG. 5.

FIG. 5 is a top plan view of the second embodiment of the self-adhering bandage of the invention.

### DETAILED DESCRIPTION OF SPECIFIC EMBODIMENTS

The present invention is a self-adhesive bandage that, upon removal, leaves an indicia on the epidermis.

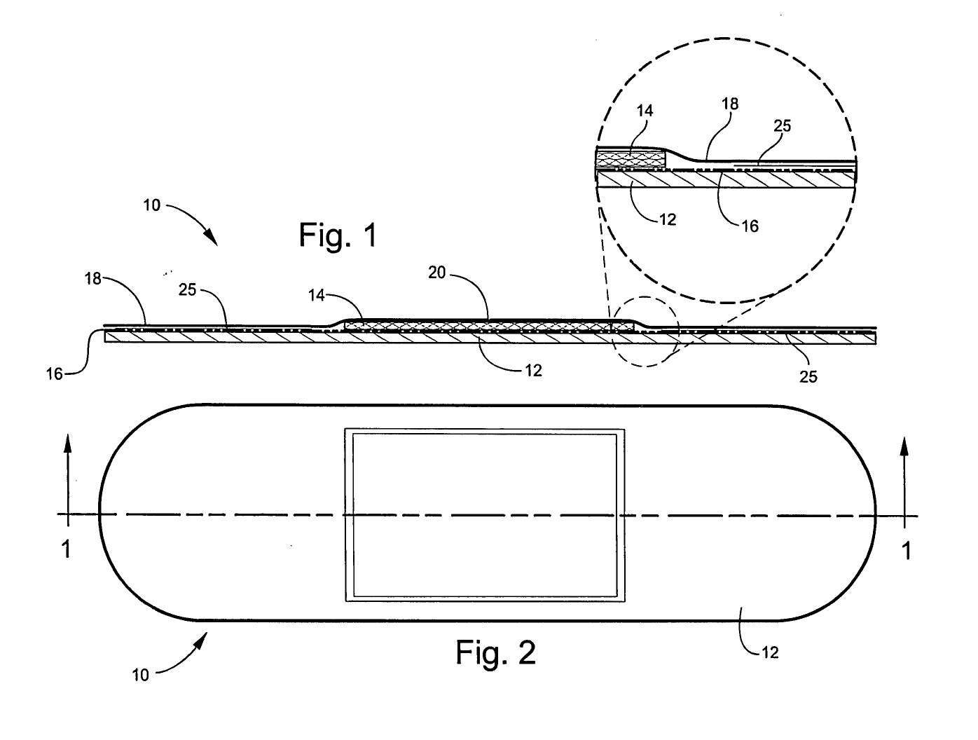

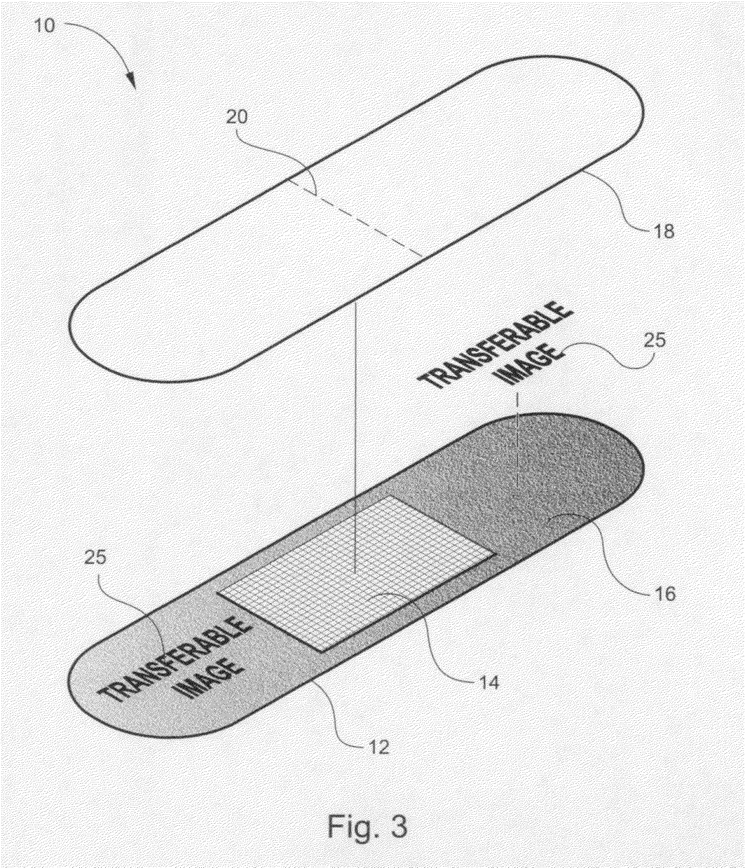

FIG. 1 is a sectional view of a self-adhering bandage, generally 10, along line 1-1 of FIG. 2. The Figure shows a carrier strip 12 having a contact or pressure sensitive adhesive layer 16 disposed thereon. Located approximately centrally on the carrier strip 12 is a gauze strip 14. Covering the gauze strip 14 is a release paper 18, held in place by its adhering contact with the pressure sensitive adhesive 16, thus forming a laminate. The release paper 18 may be scored in the center at 20 in order to facilitate its removal or it may be two separate pieces. Incorporated in the adhesive layer 16 is an image or indicia 25 that is transferable from the adhesive layer 16 and a user's skin, when the bandage 10 is removed from the user's skin.

In use, when a user has sustained an injury, such as a minor cut, scratch or abrasion, they take out a bandage 10 according to the present invention, remove the release paper 18, center the gauze strip 14 over the minor cut, scratch or abrasion and apply the carrier strip 12 to the user's skin, with the adhesive layer 16 in contact with the user's skin. In this embodiment, the adhesive layer 16 further comprises a transferable image 25, which transfers from the adhesive layer 16 to the user's skin, when the adhesive layer 16 is in direct contact with the user's skin. Thus, once the carrier strip 12, adhesive layer 16, and gauze strip 14 are removed from the user's skin, at least a portion of the transferable image 25 remains on the user's skin.

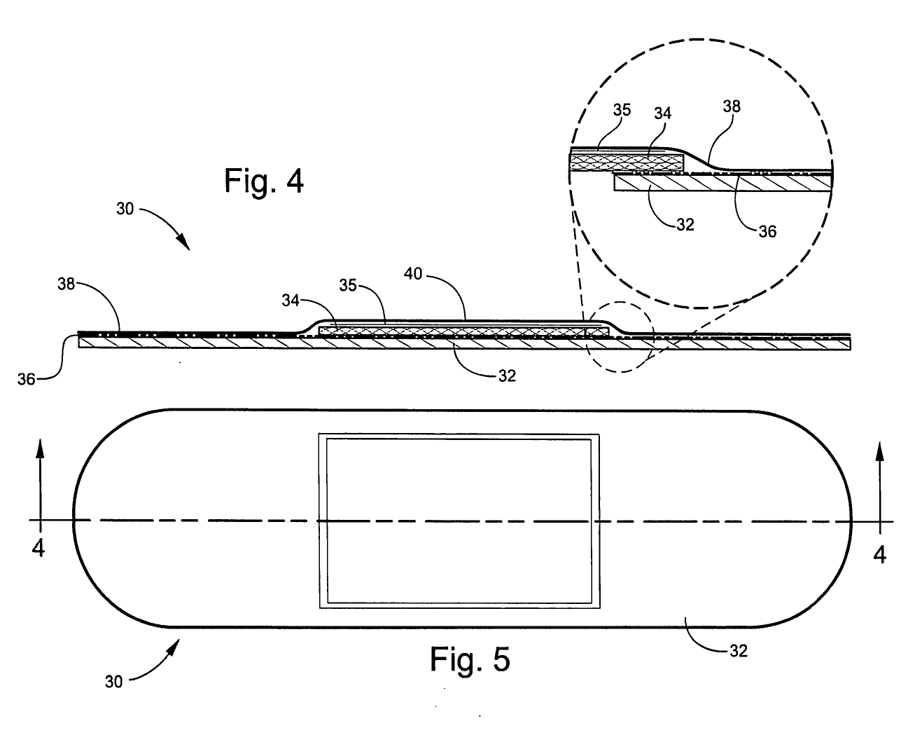

FIG. 3 is a sectional view of a second embodiment of a self-adhering bandage, generally 30, along line 3-3 of FIG. 4. The Figure shows a carrier strip 32 having a contact or pressure sensitive adhesive layer 36 disposed thereon. Located approximately centrally on the carrier strip 32 is a gauze strip 34. Covering the adhesive layer 36 is a release/image layer 35 that incorporates an image. Covering the polymer release/image layer 35 is a release paper 38, held in place by its adhering contact with the pressure sensitive adhesive 36, thus forming a laminate. The release paper 38 may be scored in the center at 40 in order to facilitate its removal or it may be two separate pieces.

In use, when a user has sustained an injury, such as a minor cut, scratch or abrasion, they take out a bandage 30 according to the present invention, remove the release paper 38, center the gauze strip 34 over the minor cut, scratch or abrasion and apply the carrier strip 32 to the user's skin, with the polymer release/image layer 35 in contact with the user's skin. In this embodiment, the polymer release/image layer 35 transfers from the adhesive layer 36 to the user's skin. Thus, once the carrier strip 32, adhesive layer 36, and gauze strip 34 are removed from the user's skin, the polymer release/image layer 35 remains on the user's skin.

The term “image or indicia” as referred to in connection with the present invention, is meant to describe the relatively thin, ink or colorant layer, which may vary considerably in consistency.

In the first embodiment, the indicia 25 is a transferable ink layer located on the surface of the adhesive layer 16 and between the adhesive layer 16 and the release paper 18. In this embodiment, the indicia layer 20 does not interfere with the adhesive properties of the adhesive layer 16, but remains behind on the skin when the bandage is removed.

In the second embodiment, the polymer release/image layer 35 is a multi-color indicia on an extremely thin, flexible, extensible film of water resistant material.

The transferable imager 25 or polymer release/image layer 35 lends itself to application to any flat surface including skin, and it closely simulates an actual tattoo by virtue of its extremely thin, flexible, extensible, matte surfaced film. A multi-color offset lithograph logo (or other design) is imprinted on the film. The printed logo comprises an extremely thin application of colored ink (or multiple applications for different colors) to the thin film.

The bandage construction will depend upon the size of the wound to be treated, the nature of the materials from which the bandage is made, and similar considerations. The bandages may be prepared individually, or they may more conveniently be manufactured in strips of adjacent bandages; the latter can then be severed to form individual bandages. The bandage strips may also be packaged in the form of rolls in dispenser boxes which allow bandages to be withdrawn in the form of a continuous “tape” and removed from the release paper to which they are attached as single bandages.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A self-adhering bandage comprising:
a. a carrier strip;
b. adhesive layer disposed thereon;
c. a gauze strip located approximately centrally on the carrier strip;
d. a release paper covering the gauze strip and held in place by its adhering contact with the adhesive, thus forming a laminate; and
e. an indicia incorporated in the adhesive layer that is transferable from the adhesive layer and a user's skin, when the bandage is removed from the user's skin.

2. The self-adhering bandage according to claim 1 wherein the indicia comprises an ink or a colorant.

3. The self-adhering bandage according to claim 2 wherein the indicia is a transferable ink layer located on the surface of the adhesive layer.

4. A self-adhering bandage comprising:
a. a carrier strip;
b. adhesive layer disposed thereon;
c. a gauze strip located approximately centrally on the carrier strip;
d. a release paper covering the gauze strip and held in place by its adhering contact with the adhesive, thus forming a laminate; and
e. a release/image layer that incorporates an image that is interposed between the adhesive layer and the release paper, said release/image layer transferable to a user's skin, when the bandage is removed from the user's skin.

5. The self-adhering bandage according to claim 1 wherein the release/image layer comprises multi-color indicia on an extremely thin, flexible, extensible film.
